(19) United States
(12) Patent Application Publication
(10) Pub. No.: US 2026/0100248 A1
(43) Pub. Date: Apr. 9, 2026

(54) AUTOMATED SYSTEMS AND METHODS FOR PATHOGEN IDENTIFICATION

(71) Applicant: Symphony Diagnostics, Inc., Stateline, NV (US)

(72) Inventors: Julius Barsi, Stateline, NV (US); Arnulf Graf, Stateline, NV (US); Joe Schroeter, Stateline, NV (US)

(21) Appl. No.: 19/352,015

(22) Filed: Oct. 7, 2025

Related U.S. Application Data
(60) Provisional application No. 63/704,334, filed on Oct. 7, 2024.

(51) Int. Cl. G16B 25/20; C12Q 1/6844; C12Q 1/6869; C12Q 1/689; C12Q 1/6893; C12Q 1/6895; C12Q 1/70; G16B 30/00; G16B 40/20; G16H 50/20

(52) U.S. Cl. CPC G16B 25/20; C12Q 1/6844; C12Q 1/6869; C12Q 1/689; C12Q 1/6893; C12Q 1/6895; C12Q 1/701; G16B 30/00; G16B 40/20; G16H 50/20

(57) ABSTRACT

Provided herein are systems and methods for pathogen identification. In particular, provided herein are methods for designing a diagnostic assay for two or more target agents utilizing artificial intelligence and machine learning (AI/ML) systems, the methods include designing amplification primer sets for one or more nucleic acid molecular identifiers for two or more targets and generating one or more collections of amplification primer sets according to their collective distinguishing power in a putative amplification assay for identifying and distinguishing any two or more or all of the two or more target agents.

### CROSS-REFERENCE TO RELATED APPLICATIONS

The present application claims priority to U.S. Provisional Patent Application Ser. No. 63/704,334, filed Oct. 7, 2024, the disclosure of which is herein incorporated by reference in its entirety.

### FIELD

Provided herein are systems and methods for pathogen identification. In particular, provided herein are methods for designing a diagnostic assay for two or more target agents utilizing artificial intelligence and machine learning (AI/ML) systems, the methods include designing amplification primer sets for one or more nucleic acid molecular identifiers for two or more targets and generating one or more collections of amplification primer sets according to their collective distinguishing power in a putative amplification assay for identifying and distinguishing any two or more or all of the two or more target agents.

### BACKGROUND

Emergent biological threats have, in recent years, accelerated both in their frequency as well as their ability to circumvent existing countermeasures. Whether due to environmental circumstances or the product of sophisticated biotechnology from negligent or bellicose foreign entities, the consequence has already been proven capable of disrupting societal order and devastating the economic landscape. Regardless of which sector is predominantly affected by any particular threat; whether it be the civilian population, military personal, agricultural (plant or livestock), aquicultural, companion animal or wildlife-the potential to cross over from one sector to another and rapidly escalate in scale constitutes a novel challenge for controlling these biological threats before and after emergence. Systems and methods to rapidly respond to a novel or emerging biological threat are lacking.

### SUMMARY

Provided herein are systems and methods for identification of pathogens, e.g., for designing a diagnostic assay for two or more target agents.

In some embodiments, provided herein are computer implemented methods for designing a diagnostic assay for one or more target agents. In some embodiments, the methods comprise: a) determining nucleic acid molecular identifiers for the one or more target agents; b) designing an amplification primer set for one or more nucleic acid molecular identifiers; c) generating one or more collections of amplification primer sets for all of the one or more target agents; d) classifying each of the one or more collections of amplification primer sets according to their collective distinguishing power in a putative amplification assay for identifying and distinguishing any one or more or all of the one or more target agents; and e) validating at least one of the one or more collections of amplification primer sets in an amplification assay.

In some embodiments, any one or more or all of steps a-d are carried out with an artificial intelligence and machine learning (AI/ML) system. In some embodiments, any one or more or all of steps a-e are automated.

In some embodiments, the one or more target agents comprises two or more target agents. In some embodiments, the one or more target agents comprises five or more target agents.

In some embodiments, determining nucleic acid molecular identifiers comprises gathering genomic sequences for each of the one or more target agents and analyzing the genomic sequences by similarity-based clustering, identification of oligonucleotide sequences directed to target agents, novelty detection, or a combination thereof.

In some embodiments, the methods further comprise classifying the amplification primer sets for the nucleic acid molecular identifiers based on the individual distinguishing power of the putative amplicons generated from the amplification primer set.

In some embodiments, generating one or more collections of amplification primer sets comprises selecting a first collection comprising a minimal collection of amplification primer set and generating subsequent collections by replacing adding and/or deleting amplification primer sets from the first collection. In some embodiments, one or more collections of amplification primer sets distinguish each of the one or more target agents.

In some embodiments, classifying each of the one or more collections of amplification primer sets comprises creating guidelines to interpret results of the putative amplification assay.

In some embodiments, validating at least one of the one or more collections of amplification primer sets comprises comparing the results of the amplification reaction to the guidelines and/or results of the putative amplification assay.

In some embodiments, the method further comprises selecting the one or more target agents. In some embodiments, at least one of the one or more target agents is a nucleic acid signature derived from a host for the purpose of a host assessment. In some embodiments, the host assessment comprises an analysis of the host's biological state or response. In some embodiments, the one or more target agents are two or more microorganisms and/or viruses. In some embodiments, the one or more microorganisms and/or viruses comprises any combination of viruses, bacteria, protozoa, algae, and fungi. In some embodiments, the one or more microorganisms and/or viruses comprise one or more pathogenic microorganisms and/or viruses. In some embodiments, the one or more microorganisms and/or viruses comprise one or more non-pathogenic microorganisms and/or viruses. In some embodiments, the one or more non-pathogenic microorganisms comprises one or more endogenous symbiotic microorganisms. In some embodiments, the one or more microorganisms and/or viruses comprises an emerging microorganism and/or virus. In some embodiments, the one or more microorganisms and/or viruses comprise two or more strains or variants of a single microorganism and/or virus.

In some embodiments, the method (e.g., determining step) comprises a simultaneous assessment of both one or more microorganisms and/or viruses and one or more host-derived signatures.

In some embodiments, the methods further comprise conducting an amplification assay on one or more samples with one or more validated collections of amplification primer sets.

In some embodiments, the methods further comprise generating a report based on the nucleic acid molecular identifiers, putative amplification assay, and amplification assay results.

In some embodiments, provided herein are systems comprising a processor running software configured to carry out any one or more or all of the steps of the method disclosed herein. In some embodiments, the system is configured to carry out each of the steps of the method disclosed herein. In some embodiments, the system further comprises a processing component. In some embodiments, the system further comprises an assay component. In some embodiments, the assay component comprises an automated nucleic acid amplification and sequencing component.

Other aspects and embodiments of the disclosure will be apparent in light of the following detailed description and accompanying figures.

### BRIEF DESCRIPTION OF FIGURES

FIG. 1 shows an exemplary flowchart of embodiments of the systems and methods described herein. Briefly, the process begins by selecting a detection group. From that point on, the systems and methods accomplish the following objectives: identify unique genomic features of each diagnostic target, design primer-pairs to extract said features, conceive of rules by which to leverage all of the unique features, generate a final report based on the results of the assay, analyze the performance, and extract all meaningful data derivable from the unique features, which are of commercial value.

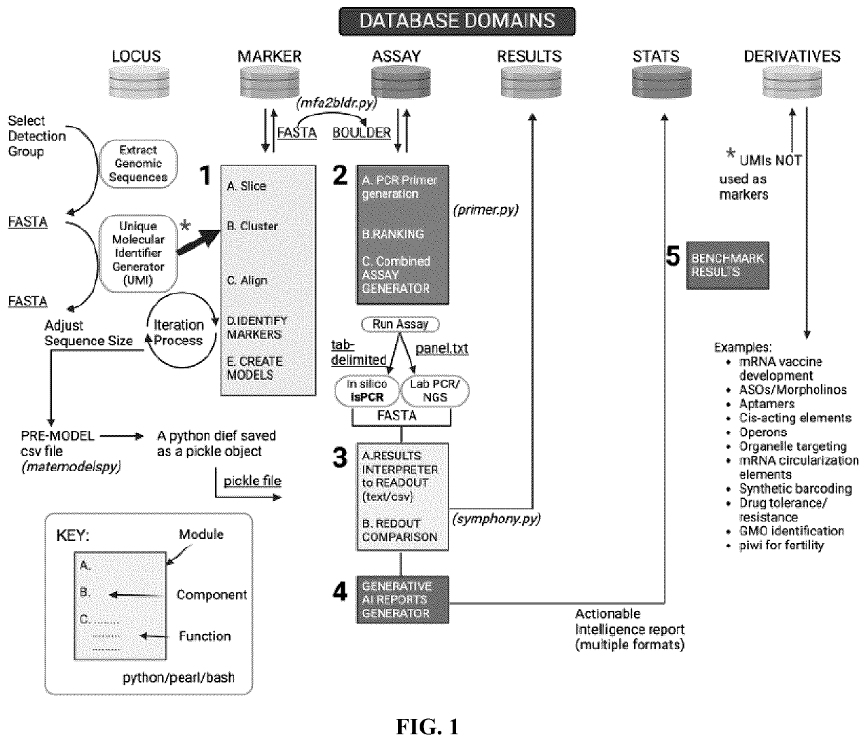

FIG. 2 shows an embodiment of a multiplex PCR medicated sequencing assay suitable for use with the disclosed systems and methods. A plurality of pathogenic agents can be interrogated in a single sample in a high-throughput automated coupled to amplicon sequences and real-time analytics.

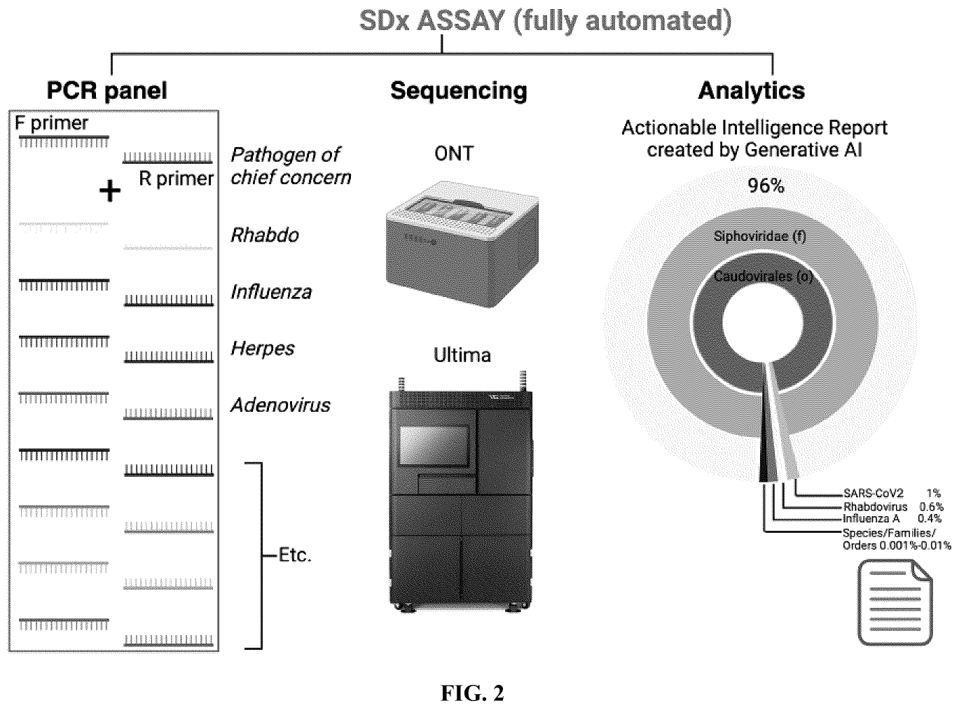

FIG. 3 shows as schematic of an exemplary analysis of SARS-COV-2. Select genomic regions are amplified and sequenced enabling high-throughput and automated variant detection currently not accomplished by routine detection and identification methods.

### DETAILED DESCRIPTION

The disclosed systems and methods facilitate pathogen target identification which enables a rapid response to a novel or emerging biological threat. The disclosed systems and methods utilize a variety of tools to facilitate the gathering of detailed information on newly sequenced organisms, the features of their genome most likely to serve for the development of countermeasures, and a variety of analytical tools that captures the broad implications so often lost in most diagnostic approaches. The disclosed systems and methods provide a fully automated platform for the rapid design of diagnostic assays capable of identifying pathogens. The disclosed systems and methods provide a generalizable process that can be applied to pathogen, particularly emerging biological threats.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “oligonucleotide,” as used herein, refers to a short nucleic acid sequence comprising from about 2 to about 100 nucleotides (e.g., about 5, 10, 15, 20, 25, 30, 35, 40, 45, 50, 55, 60, 65, 70, 75, 80, 85, 90, 95, 99, or 100 nucleotides, or a range defined by any of the foregoing values). The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double-and single-stranded DNA, and double-and single-stranded RNA. The terms include, as equivalents, analogs of either RNA or DNA made from nucleotide analogs and modified polynucleotides such as, for example, methylated and/or capped polynucleotides. Nucleic acids are typically linked via phosphate bonds to form nucleic acid sequences or polynucleotides, though many other linkages are known in the art (e.g., phosphorothioates, boranophosphates, and the like).

As used herein, the term “computer” refers to a machine, apparatus, or device that is capable of accepting and performing logic operations from software code. The term “application,” “software,” “software code,” or “computer software” refers to any set of instructions operable to cause a computer to perform an operation. Software code may be operated on by a “rules engine” or “processor.” Thus, in some embodiments, the methods and systems of the present invention may be performed by a computer or computing device having a processor based on instructions received by computer applications and software.

The term “electronic computer device” as used herein, is a type of computer comprising circuitry and configured to generally perform functions such as recording and analyzing data; generating, formatting, and analyzing databases; generating reports; storing, retrieving, or manipulation of electronic data; providing electrical communications and network connectivity; or any other similar function. Non-limiting examples of electronic devices include: personal computers (PCs), workstations, laptops, tablet PCs including the iPad, cell phones including iOS phones made by Apple Inc., Android OS phones, Microsoft OS phones, Blackberry phones, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”.

The term “computer readable medium” as used herein refers to any medium that participates in providing instructions to the processor for execution. A computer readable medium may take many forms, including but not limited to, non-volatile media, volatile media, and transmission media. Non-volatile media includes, for example, optical, magnetic disks, and magneto-optical disks, such as the hard disk or the removable media drive. Volatile media includes dynamic memory, such as the main memory. Transmission media includes coaxial cables, copper wire and fiber optics, including the wires that make up the bus. Transmission media may also take the form of acoustic or light waves, such as those generated during radio wave and infrared data communications. Non-transitory computer readable media includes all computer readable media, with the sole exception being a transitory, propagating signal per se.

As used herein the term “data network” or “network” shall mean an infrastructure capable of connecting two or more computers such as client devices either using wires or wirelessly allowing them to transmit and receive data. Non-limiting examples of data networks may include the Internet or wireless networks which may include Wi-Fi and cellular networks. For example, a network may include a local area network (LAN), a wide area network (WAN) (e.g., the Internet), a mobile relay network, a metropolitan area network (MAN), an ad hoc network, a telephone network (e.g., a Public Switched Telephone Network (PSTN)), a cellular network, a Zigby network, or a voice-over-IP (VOIP) network.

As used herein, the term “database” shall generally mean a digital collection of data or information. For the purposes of the present disclosure, a database may be stored on a remote server and accessed by a client device (e.g., through the Internet) or alternatively in some embodiments the database may be stored on the client device or remote computer itself.

As used herein, the term “artificial intelligence” shall generally mean smart machines capable of performing tasks that typically require human-like intelligence and the machines learning from experience, adjusting to new inputs, processing large amounts of data, and recognizing patterns in the data.

As used herein, the term “machine learning” shall generally mean smart machines using statistics to find patterns in large amounts of data, wherein the data is anything that can be digitally stored. Machine learning is seen as a subset of artificial intelligence, and machine learning algorithms make predictions based on data without being programmed to specifically do so.

As used herein, the term “deep learning” is a subset of machine learning that uses artificial neural networks with a large number of hidden layers. Such networks were designed to simulate brain-like processing of complex information, for example, to progressively extract higher level features from raw data input. These networks can comprise convolutional as well as recurrent networks.

The present disclosure provides automated systems and methods related to agent identification. In particular, the present disclosure provides systems and methods for automated design of an assay (e.g., amplification primer sets and collections of amplification primer sets) for one or more target agents.

In some embodiments, the methods comprise determining nucleic acid molecular identifiers for one or more target agents. Nucleic acid molecular identifiers include those sequences capable of distinguishing an individual target from all other targets (e.g., in the same assay). Thus, nucleic acid molecular identifiers are unique sequences to a target agent that allows it to be identified and/or distinguished from other potential target agents in an assay. The nucleic acid molecular identifies are limited by sequence or length.

In some embodiments, determining nucleic acid molecular identifiers comprises gathering genomic sequences for each of the one or more target agents and analyzing the genomic sequences by similarity-based clustering, identification of oligonucleotide sequences (K-mers) directed to target agents, novelty detection, or a combination thereof. Any of these techniques can be used individually or in combination, e.g., ensembled together with machine learning techniques, such as hard or soft voting scheme.

For similarity-based clustering, the genomic sequence of each target agent is partitioned into overlapping fragments. The size of each partition and the degree of overlap are two parameters of bioinformatic import. In order to accommodate a variety of agents with dissimilar replication schemes, a scaled approach can be used by combining different fragmentation strategies.

Nucleic-acid sequences are clustered by comparing two datasets at a time, using an ultrafast method based on short word filtering. Briefly, the minimal number of identical short substrings termed ‘words’, such as di-nucleotides, tri-nucleotides, etc. which are shared by two genomes is a function of their sequence similarity. This function is computed using analytical and statistical analyses. As a result it is possible to estimate that the similarity of two sequences is below a certain threshold by straight-forward word counting, avoiding a sequence alignment. This may be implemented through the use of an index table, lessening the computational operations required. A clustering algorithm arranges sequences according to their length. The longest sequence becomes the representative of the first cluster. Subsequently, each remaining sequence is compared with the representatives of existing clusters. Should the similarity with any representative lie above a particular threshold, it becomes a member of that cluster. If the sequence similarity is below a particular threshold, a new cluster is defined with that sequence as the representative. At each step, short word filtering can be used to confirm that the similarity falls below the clustering threshold. If the confirmation fails, sequence alignments may be used to complete the confirmation.

The desired result of similarity-based clustering is to attain stretches of unique sequence that do not cluster and can therefore be used as a signature for the genome of a target agent. The process is repeated by comparing the putative molecular identifiers to the genome of interest until the genomes of all the one or more target agents have been evaluated in this manner.

Identification of oligonucleotide sequences directed to target agents is based on the creation of K-mers, where K is the length of a sequence. K-mers are subsequences of the entire sequence. For example, when K=1, there are four DNA K-mers; A, T, G, and C. The “K” value is tunable for both target and non-target sequences. The K-mers for any sequence are stowed in native Go maps, analogous to a key/value dictionary in Python. Subsequently, target and non-target maps are cross-referenced in order to generate a unique set of K-mers that are absent from the non-target map, e.g., only found in the target sequence. Target specific K-mers may be matched their original position within the entire sequence in order to locate regions of overlapping K-mers that are merged to create larger molecular identifiers.

Identification of unique sequences can be done using novelty detection. In some embodiments, novelty detection is completed by a machine learning (ML) approach. ML-based novelty detection is a form of semi-supervised learning where the training data only contains regular data (e.g., no novel patterns). Novelty detection then estimates whether a new observation is a novelty, outlier, as compared to the regular data. Novelty detection does not require labels associated with the data, contrary to classification or regression, and is insensitive to class imbalance. The input can be a collection of genomes that represent the target agents. The number of target agents is not limited. In some embodiments, the input comprises multiple genomes per target agent to account for genetic variability. In some embodiments, the input includes 1 to 20 genomes per pathogen. In some embodiments, the input includes more than 20 genomes per pathogen. The novelty of a catalog of sequence strings created from the genomes can be predicted and serve as a proxy by which to identify and distinguish the agents. The sequence strings are not limited by length. In some embodiments, the sequence strings are 50-5000 base pairs in length. In some embodiments, the sequence strings are greater than 5000 base pairs in length.

In some embodiments, the methods comprise designing an amplification primer set for one or more nucleic acid molecular identifiers. The nucleic acid molecular identifiers can serve as template DNA sequence for the design of primer sets for the purpose of amplification reactions. The amplification primer sets are designed to include and amplify the majority or all of the nucleic acid molecular identifier. In some embodiments, primer design includes any one or all of: thermodynamic models for predicting primer melting temperatures; primer binding; and the formation of primer dimers and/or secondary structure. Factoring in these characteristics of any one primer sequence facilitates design of the primers around a variety of constraints, e.g., maximum amplicon length relative to the nucleic acid molecular identifier. In some embodiments, a search-and-avoid strategy is implemented to minimize cross-reactive primer pairs. In some embodiments, primer characteristics and conditions are those suitable for multi-plex reactions.

As used herein, the terms “primer set” and “amplification primer set” refer to two or more oligonucleotides which together are capable of priming the amplification of a target sequence or target nucleic acid of interest. In certain embodiments, the terms “primer set” and “amplification primer set” refer to a pair of oligonucleotides including a first oligonucleotide that hybridizes with the 5′-end of the target sequence or target nucleic acid to be amplified and a second oligonucleotide that hybridizes with the complement of the target sequence or target nucleic acid to be amplified.

The term “primer” as used herein, refers to an oligonucleotide which is capable of acting as a point of initiation of synthesis of an extension product that is a complementary strand of nucleic acid (all types of DNA or RNA) when placed under suitable amplification conditions (e.g., buffer, salt, temperature and pH) in the presence of nucleotides and an agent for nucleic acid polymerization (e.g., a DNA-dependent or RNA-dependent polymerase). The primers of the present disclosure can be of any suitable size, and desirably comprise, consist essentially of, or consist of about 15 to 50 nucleotides, preferably about 20 to 40 nucleotides.

In some embodiments, the methods further comprise classifying the amplification primer sets for the nucleic acid molecular identifiers based on the individual distinguishing power of the putative amplicons generated from the amplification primer set.

In some embodiments, the classifying is completed using machine learning discriminators that classify a sequence as a molecular identifier or not, e.g. these are termed “tailings”. A feature engineering algorithm based on probabilistic models of N-uples uses a confusion matrix from these classifications as a discrimination metric. This facilitates finding the most accurate classification model and benchmark the results against those previously published. The classifier can be used to rank and score the features of the sequence. For this, a recursive ranking framework based on the discrimination metric can be used. Because the data is subject to class imbalance, standard machine learning classifiers are not suitable, including ranking and scoring. Instead of under-sampling, which would ignore most of the over-represented data, a bagging scheme can be applied to the classifiers, thus “crating” an optimized ensemble of (balanced) classifiers operating on equal data subsets.

In some embodiments, the methods comprise generating one or more collections of amplification primer sets for all of the one or more target agents. The one or more collections of amplification primer sets distinguish each of the one or more target agents from each other. The collection of amplification primer sets facilitates identifying and distinguishing all target agents in a single assay, e.g., a multiplex reactions.

In some embodiments, generating the collections of amplification primer sets comprises selecting a first collection comprising a minimal collection of amplification primer set and generating subsequent or additional collections by replacing adding and/or deleting amplification primer sets from the first collection. This process can be repeated by combinatorial selection until all possible collections of the amplification primer sets are generated.

During a standard experimental validation process, any issue that may arise in the experimental validation undergoes trouble shooting, then the assay design is amended and undergoes another round of experimental validation. This recursive pattern is followed until a final assay performs as expected. The methods and systems described herein, circumvent this by providing a plethora of alternative versions of the same assay. These are ranked according to their discriminatory power and other factors such as cost/performance ratio. At the top of the catalog, the version with the highest recommendation undergoes experimental validation first. Should an issue arise during this process, no additional trouble shooting is undertaken, Rather, the design is discarded and the next ranking alternative version is used. Eventually, the design that has no issue during experimental validation will then become the viable product.

In some embodiments, the methods comprise classifying each of the one or more collections of amplification primer sets according to their collective distinguishing power in a putative amplification assay for identifying and distinguishing any one or more or all of the one or more target agents. A putative amplification assay, e.g., an in silico PCR, predicts the sequence of the amplicons generated for each amplification primer set. These predicted amplicons can then be used to characterize the collection by how the amplicons can distinguish all the target agents, and in the most efficient manner (e.g., with the fewest amplification primer sets in the assay), and how each amplicon benefits the collective distinguishing power. These predicted amplicons are also validated against multiple reference genomes for each organism or diagnostic target, and for no cross talk with any genomes from primate, avian, ungulate, ruminant, or porcine organisms. Machine learning techniques can be used to combine features from the minimal set (features engineering) and then train a linear classifier on these. The separating hyperplane is then a proxy for how each amplicon benefits the collective distinguishing power.

In some embodiments, classifying each of the one or more collections of amplification primer sets comprises creating guidelines to interpret results of the putative amplification assay. For example, if there are 25 markers for a particular diagnostic target, one guideline may state that markers 1-5 must be amplified to consider the pathogen in question as “present” in the sample. Whereas the amplification of the other markers informs which variant is present. The guideline might refer to marker 6 & 7 having to simultaneously be amplified in order to call out one variant, or a guideline may say that the sequence content of marker 8 match the sequence on file. The ultimate goal is to assemble the necessary primer pairs to extract the desired information, and the rules are developed for their ability to provide the desired information which the assay tests for.

For each amplification primer set, henceforth referred to as putative-assays, guidelines/rubrics can be specified on a case-by-case basis for the desired agents and assay. This process may be automated by the use of machine learning. ML models scale automatically with increasing number of features when the model is re-trained, akin to transfer learning for deep learning when only the last layer of the ANN (artificial neural network) is retrained.

Identified collections of amplification primer sets which meet the guidelines/rubrics for the putative amplification assay may then be validated in an in an amplification assay. Thus, in some embodiments, the methods comprise validating at least one of the one or more collections of amplification primer sets in an amplification assay. One or more collections of amplification primer sets may be validated concurrently. Alternatively, a single amplification primer set may proceed for validation and if the result is not as expected or there is a need for alternatives, subsequence collections of amplification primer sets can be additionally validated, e.g., until desired result is achieved, as compared to the guidelines/rubrics for the putative amplification assay.

Once a collection of amplification primer sets is validated, the molecular identifiers generated during the method can be used to create derivatives. Generative AI and variants of reinforcement learning can be used to create novel derivatives using derivative databases. The process may cross reference any or all unused molecular identifiers against large datasets. The derivative databases could provide a repository for sequences or downstream applications of future interest, including, for example, a catalog of novel sequences, genomic coordinates, or a dynamic window that could scan sequence and in so doing reveal features/value.

In some embodiments, any or all of the steps of the above disclosed method are carried out with an artificial intelligence and machine learning (AI/ML) system. In some embodiments, each of: determining nucleic acid molecular identifiers for the one or more target agents; designing an amplification primer set for one or more nucleic acid molecular identifiers; generating one or more collections of amplification primer sets for all of the one or more target agents; and classifying each of the one or more collections of amplification primer sets according to their collective distinguishing power in a putative amplification assay for identifying and distinguishing any one or more or all of the one or more target agents are carried out with artificial intelligence and machine learning (AI/ML) system(s). In some embodiments, each of the listed steps are fully automated and are combined into a single AI/ML system.

These generated collections of amplification primer sets can then be used to analyze samples, e.g., in an amplification assay. Thus, in some embodiments, the methods comprise conducting an amplification assay on one or more samples with one or more validated collections of amplification primer sets. Any amplification assay may comprise both amplification and sequencing of the generated amplicons.

Illustrative non-limiting examples of nucleic acid amplification techniques include, but are not limited to, polymerase chain reaction (PCR), TAQMAN amplification, reverse transcription polymerase chain reaction (RT-PCR), transcription-mediated amplification (TMA), ligase chain reaction (LCR), strand displacement amplification (SDA), and nucleic acid sequence-based amplification (NASBA). Those of ordinary skill in the art will recognize that certain amplification techniques (e.g., PCR) typically involve RNA reverse transcription to DNA prior to amplification (e.g., RT-PCR), whereas other amplification techniques directly amplify RNA (e.g., TMA and NASBA).

Suitable nucleic acid sequencing techniques include, but are not limited to, sequencing by synthesis (see e.g., Meyer and Kircher, “Illumina sequencing library preparation for highly multiplexed target capture and sequencing,” Cold Spring Harbor Protocols 2010 (6)); single-molecule real-time sequencing (see e.g., Levene et al., “Zero-Mode Waveguides for Single-Molecule Analysis at High Concentrations,” Science. 299(5607): 682-6 (2003)); ion semiconductor sequencing (see e.g., Rusk, “Torrents of sequence,” Nat. Methods 8, 44 (2011)); pyrosequencing (see e.g., Wicker et al., “454 sequencing put to the test using the complex genome of barley,” BMC Genomics, 7:275, 2006); sequencing by ligation (SOLiD sequencing) (see e.g., Margulies et al., “Genome sequencing in microfabricated high-density picolitre reactors,” Nature, 437:376-80 (2005)); nanopore sequencing (see e.g., Goodwin et al., “Oxford Nanopore sequencing, hybrid error correction, and de novo assembly of a eukaryotic genome,” Genome Res., 25(11):1750-6 (2015)); chain termination sequencing (Sanger sequencing) (see e.g., Sanger et al., “DNA sequencing with chain-terminating inhibitors,” Proceedings of the National Academy of Sciences of the United States of America, 74 (12): 5463-5467 (1977)); and sequencing with mass spectrometry (see e.g., Edwards et al., “Mass-spectrometry DNA sequencing,” Mutation Research, 573(1-2): 3-12 (2005)). The use of Oxford Nanopore Technology allows for the obtaining of >10 kb uninterrupted sequences—enough to resolve whole plasmids (harboring antimicrobial resistance genes) and the phylogenetic relationship of new emerging variants of microorganisms or viruses relative to existing ones circulating through the population.

In some embodiments, the sample is a non-human sample. For example, the sample may be from a non-human animal (e.g., a farm animal, a companion animal, a wild animal, an aquatic animal, an animal in captivity, an endangered or threatened species of animal). In some embodiments, the sample is from a human.

In some embodiments, the sample is from a farm animal. In some embodiments, the farm animal is selected from the group consisting of dairy cattle, sheep, horses, goats, chickens, pigs, rabbits, deer, turkeys, mules, banteng, boars, bison, beef cattle, emu, donkeys, geese, camels, reindeer, pheasants, ducks, quails, domestic yaks, llamas, American pygmies, alpacas, ostrich, elk, and fish. In some embodiments, the animal is a companion animal. In some embodiments, the companion animal is a dog, cat, horse, rabbit, ferret, bird, guinea pig, fish turtle, snake, or lizard. In some embodiments, the animal is a wild animal. In some embodiments, the wild animal is a lion, tiger, leopard, cheetah, jaguar, elephant, giraffe, hippopotamus, rhinoceros, gorilla, chimpanzee, orangutan, bear, wolf, coyote, fox, lynx, bobcat, mountain lion, zebra, wildebeest, gazelle, antelope, warthog, hyena, jackal, crocodile, alligator, turtle, snake, kangaroo, koala, wombat, wallaby, platypus, octopus, squid, crab, lobster, shrimp, clam, oyster, snail, walrus, seal, whale, dolphin, manatee, skink, lizard, gecko, chameleon, bat, raccoon, opossum, rat, mouse, chipmunk, rabbit, badger, skunk, armadillo, porcupine, beaver, otter, seagull, eagle, flacon, hawk, osprey, vulture, owl, parrot, heron, swan, goose, duck, ostrich, turkey, emu, camel, llama, yak, deer, moose, caribou, bison, buffalo, or elk. In some embodiments, the animal is an aquatic animal. In some embodiments, the aquatic animal is a carp, pollock, clam, tilapia, shrimp, tuna, anchovy, salmon, herring, mackerel, rohu, cod, squid, trout, crab, sardine, haddock, catfish, eel, scallop, prawn, shark, perch, albacore, or bass. In some embodiments, the animal is an endangered or threatened species.

In some embodiments, the sample is derived from a human or non-human animal having or suspected of having a disease or disorder. In some embodiments, the sample is derived from a human or non-human animal having or suspected of having a disease or disorder mediated by one or more microorganisms or viruses. In some embodiments, the sample is derived from a healthy human or non-human animal.

The sample can be obtained from the subject using routine techniques known to those skilled in the art, and the sample may be used directly as obtained from the biological source or following a pretreatment to modify the character of the sample. Such pretreatment may include, for example, preparing plasma from blood, diluting viscous fluids, filtration, precipitation, dilution, distillation, mixing, concentration, inactivation of interfering components, the addition of reagents, lysing, and the like.

In some embodiments, the methods comprise generating a report based on the nucleic acid molecular identifiers, putative amplification assay, and amplification assay results. Report generation can be manual or automated. In some embodiments, generative artificial intelligence is used to create actionable intelligence reports based on the results for the nucleic acid molecular identifiers and from the putative amplification assay and amplification assay (e.g., the guidelines/rubrics and validation results).

To interpret and communicate results from a single amplification reaction based on the disclosed methods, Large Language Models (LLMs) can be trained with the results and a chatbot can create an intelligent report. For example, the LLMs may be trained on the test results, for example, from a set list of target agents being queried, a combination are present and the others absent from a sample. Using prompt engineering, the LLMs then report which are present using precise and measured language and integrate a variety of metadata pertinent to the pathogens detected. These reports will also include relative abundance of each pathogen, have different lexicons targeting each client base, for instance, military terminology vs public health vs veterinary diagnostics.

In some embodiments, the methods comprise selecting one or more target agents for which a desired assay or identification is needed. In some embodiments, one or more target agents are selected such that a single multiplex reaction may distinguish all of the one or more target agents and allow identification in a sample. For example, one or more target agents may comprise at least two, at least three, at least four, at least 5, at least 10, at least 20 different target agents being identified, distinguished, or quantified in a single sample.

The one or more target agents may be selected to be assessed in unison due to their commercial value, scientific interest or co-emergent threat status. In these instances, genomic information is available and imported into the disclosed systems and methods.

The one or more target agents may be selected based on a prior or existing assay, e.g., an assay generated by the disclosed systems and methods. The cumulative data generated by a previously developed assay can be used to improve or amend the assay according to the results encountered for end-users of the amplification assay. For example, if the assay was originally designed to detect 100 pathogens, of which 10 were never encountered by end-users of the assay, those amplification primer sets can be considered for removal or replacement. Furthermore, sequences from other target agents (e.g., variants of one or more target agents) may be identified during use of the amplification assay. In these instances, genomic information is derived from the results of the prior amplification assays and imported into the disclosed systems and methods.

In some embodiments, the one or more target agents are one or more microorganisms and/or viruses. Microorganisms include bacteria, protozoa, algae, and fungi. In some embodiments, the one or more microorganisms and/or viruses comprises any combination of viruses, bacteria, protozoa, algae, and fungi.

In some embodiments, the one or more microorganisms and/or viruses comprise one or more pathogenic microorganisms and/or viruses. In some embodiments, the one or more microorganisms and/or viruses comprises one or more non-pathogenic microorganisms and/or viruses. In some embodiments, the one or more non-pathogenic microorganisms comprises one or more endogenous symbiotic microorganisms. In some embodiments, the one or more endogenous symbiotic microorganisms comprises one or more gut flora microorganisms.

In some embodiments, a component of the systems and methods are assay kits that facilitate the analysis of a sample comprising the two or more target agents, or nucleic acids thereof. In some embodiments, the assay kits comprise multiplex reaction devices (e.g., multi-well plates) and reagents (e.g., dNTPs, buffers). In some embodiments, samples undergo a processing and pre-purification step prior to nucleic acid amplification. For example, in some embodiments, samples may undergo cellular lysis, dilution, or concentration. In some embodiments, nucleic acid is purified way from non-nucleic acid components of the sample, by, for example, capture, centrifugation, filtration, or the like.

In some embodiments, a component of the systems is hardware. In some embodiments, the hardware comprises automated sample and liquid handling components that orchestrate processing of collected samples through optional sample pre-purification steps, through assay kit sample processing, and through nucleic acid analysis and data collection. For the latter, in some embodiments, the systems and methods comprise a nucleic acid sequencer that determines target sequences from the amplified nucleic acids generated by the assay kits. Nucleic acid may be analyzed using a variety of techniques including but not limited to: nucleic acid sequencing, nucleic acid hybridization, nucleic acid amplification, and mass spectroscopy.

In some embodiments, robotic sample and liquid handling according to sample input track the progress of each sample. For instance, computer vision is used to optimize the sample and liquid handling of the robot, and track the evolution of each sample.

In some embodiments, the technology described herein is associated with a programmable machine designed to perform a sequence of arithmetic or logical operations as provided by the methods described herein. For example, some embodiments of the technology are associated with (e.g., implemented in) computer software and/or computer hardware. In one aspect, the technology relates to a computer comprising a form of memory, an element for performing arithmetic and logical operations, and a processing element (e.g., a microprocessor) for executing a series of instructions (e.g., a method as provided herein) to read, manipulate, and store data.

In some embodiments, the various embodiments of the present disclosure are associated with a plurality of programmable devices that operate in concert to perform a method as described herein. For example, in some embodiments, a plurality of computers (e.g., connected by a network) may work in parallel to collect and process data, e.g., in an implementation of cluster computing or grid computing or some other distributed computer architecture that relies on complete computers (with onboard CPUs, storage, power supplies, network interfaces, etc.) connected to a network (private, public, or the internet) by a conventional network interface, such as Ethernet, fiber optic, or by a wireless network technology.

For example, some embodiments provide a computer that includes a computer-readable medium. The embodiment includes a random-access memory (RAM) coupled to a processor. The processor executes computer-executable program instructions stored in memory. Such processors may include a microprocessor, an ASIC, a state machine, or other processor, and can be any of a number of computer processors, such as processors from Intel Corporation of Santa Clara, California and Motorola Corporation of Schaumburg, Illinois. Such processors include, or may be in communication with, media, for example computer-readable media, which stores instructions that, when executed by the processor, cause the processor to perform the steps described herein.

Computers are connected in some embodiments to a network. Computers may also include a number of external or internal devices such as a mouse, a CD-ROM, DVD, a keyboard, a display, or other input or output devices. Examples of computers are personal computers, digital assistants, personal digital assistants, cellular phones, mobile phones, smart phones, pagers, digital tablets, laptop computers, internet appliances, and other processor-based devices. In general, the computers related to aspects of the technology provided herein may be any type of processor-based platform that operates on any operating system, such as Microsoft Windows, Linux, UNIX, Mac OS X, etc., capable of supporting one or more programs comprising the technology provided herein. Some embodiments comprise a personal computer executing other application programs (e.g., applications). The applications can be contained in memory and can include, for example, a word processing application, a spreadsheet application, an email application, an instant messenger application, a presentation application, an Internet browser application, a calendar/organizer application, and any other application capable of being executed by a client device. All such components, computers, and systems described herein as associated with the technology may be logical or virtual.

In some embodiments, the systems and methods employ system control hardware and software that manages the hardware, that processes and analyzes sequences, sample metadata, and attributes, and selects sequences based on intent-specific criteria. In some embodiments, software is run on a computer processor. In some embodiments, the system and system software is multilayered, modular and scalable. In some embodiments, the software manages one or more of the following system/method operations and features: collection of sample metadata associated with each sample; collect, compile and organize all sequences and sample metadata; leverage machine learning for a variety of purposes, including the inference of non-obvious attributes; selection of sequences; and other operations and feature of the disclosed methods. In some embodiments, the systems and methods perform one or more or all of the above concomitantly and in real-time.

An artificial intelligence system component may comprise or function as artificial intelligence logic stored in memory that may be executable by a processor of one or more servers and/or client devices. In some embodiments, the artificial intelligence component may function as or comprise a machine/deep learning/artificial intelligence platform that interrogates the information or data of the system and learns about trends associated with data obtained from analysis of one or more samples, sample metadata, and information in public or private databases. The artificial intelligence component consistently undergoes algorithm testing and validation based on new data available.

### EXAMPLES

### Example 1

### Variant Detection

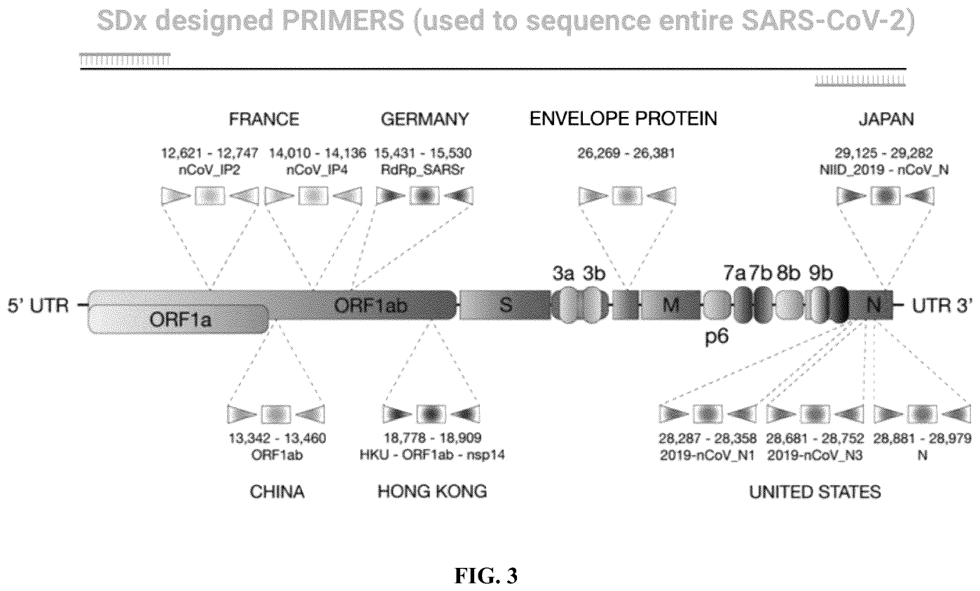

In this example, targeted amplification utilizing a primer set as designed by the disclosed methods coupled to genomic sequencing enables novel variant detection ignored by legacy diagnostic approaches. The legacy assays only interrogate a small portion of the SARS-COV-2 genome, as indicated in FIG. 3. By contrast, the SDx designed primers shown at the top of the figure (although not drawn to scale) encompass the entirety of the SARS-CoV-2 genome and interrogate each and every nucleotide. Thereby, emergent variants, e.g., particularly those which fall outside of the small portions interrogated by the legacy assays, do not elude the detection capability.

1. A computer implemented method for designing a diagnostic assay for one or more target agents, the method comprising:
a) determining nucleic acid molecular identifiers for the one or more target agents;
b) designing an amplification primer set for one or more nucleic acid molecular identifiers;
c) generating one or more collections of amplification primer sets for all of the one or more target agents;
d) classifying each of the one or more collections of amplification primer sets according to their collective distinguishing power in a putative amplification assay for identifying and distinguishing any one or more or all of the one or more target agents; and
e) validating at least one of the one or more collections of amplification primer sets in an amplification assay,
wherein any one or more or all of steps a-d are carried out with an artificial intelligence and machine learning (AI/ML) system.

2. The method of claim 1, wherein any one or more or all of steps a-e are automated.

3. (canceled)

4. The method of claim 1, wherein the one or more target agents comprises five or more target agents.

5. The method of claim 1, wherein determining nucleic acid molecular identifiers comprises gathering genomic sequences for each of the one or more target agents and analyzing the genomic sequences by similarity-based clustering, identification of oligonucleotide sequences directed to target agents, novelty detection, or a combination thereof.

6. The method of claim 1, further comprising classifying the amplification primer sets for the nucleic acid molecular identifiers based on the individual distinguishing power of the putative amplicons generated from the amplification primer set.

7. The method of claim 1, wherein generating one or more collections of amplification primer sets comprises selecting a first collection comprising a minimal collection of amplification primer set and generating subsequent collections by replacing adding and/or deleting amplification primer sets from the first collection.

8. The method of claim 1, wherein the one or more collections of amplification primer sets distinguish each of the one or more target agents.

9. The method of claim 1, wherein classifying each of the one or more collections of amplification primer sets comprises creating guidelines to interpret results of the putative amplification assay.

10. The method of claim 9, wherein validating at least one of the one or more collections of amplification primer sets comprises comparing the results of the amplification reaction to the guidelines and/or results of the putative amplification assay.

11. The method of claim 1, wherein the method further comprises selecting the one or more target agents.

12. The method of claim 11, wherein at least one of the one or more target agents is a nucleic acid signature derived from a host for the purpose of a host assessment.

13. The method of claim 12, wherein the host assessment comprises an analysis of the host's biological state or response.

14. The method of claim 11, wherein the one or more target agents are two or more microorganisms and/or viruses.

15. (canceled)

16. (canceled)

17. The method of claim 14, any wherein the one or more microorganisms and/or viruses comprise one or more non-pathogenic microorganisms and/or viruses.

18. The method of claim 17, wherein the one or more non-pathogenic microorganisms comprises one or more endogenous symbiotic microorganisms.

19. (canceled)

20. The method of claim 14, wherein the one or more microorganisms and/or viruses comprise two or more strains or variants of a single microorganism and/or virus.

21. The method of claim 1, wherein the determining comprises a simultaneous assessment of both one or more microorganisms and/or viruses and one or more host-derived signatures.

22. The method of claim 1, further comprising conducting an amplification assay on one or more samples with one or more validated collections of amplification primer sets.

23. The method of claim 1, further comprising generating a report based on the nucleic acid molecular identifiers, putative amplification assay, and amplification assay results.

24. A system comprising a processor running software configured to carry out any one or more or all of the steps of the method of claim 1.

25.-28. (canceled)
